(19) United States
(12) Patent Application Publication
(10) Pub. No.: US 2025/0194946 A1
(43) Pub. Date: Jun. 19, 2025

(54) SYSTEM, METHOD AND COMPUTER-ACCESSIBLE MEDIUM FOR DIRECT VISUALIZATION WITH POWER SPECTRUM REGULARIZATION

(71) Applicant: NEW YORK UNIVERSITY, New York, NY (US)

(72) Inventors: Tim Shepherd, Tarrytown, NY (US); Dmitry NOVIKOV, New York, NY (US); Benjamin ADES-ARON, Brooklyn, NY (US)

(21) Appl. No.: 18/942,745

(22) Filed: Nov. 10, 2024

Related U.S. Application Data
(63) Continuation of application No. PCT/US2023/021725, filed on May 10, 2023.
(60) Provisional application No. 63/340,391, filed on May 10, 2022.

(51) Int. Cl. A61B 5/055; A61B 5/00; G06T 5/60; G06T 5/70; G06T 5/73

(52) U.S. Cl. CPC A61B 5/055; A61B 5/0042; A61B 5/7203; A61B 5/7267; G06T 5/60; G06T 5/70; G06T 5/73; A61B 2576/026; G06T 2207/10088; G06T 2207/20081; G06T 2207/20084; G06T 2207/30016

(57) ABSTRACT

Exemplary system, method and computer accessible medium can be provided for creating or providing a (e.g., direct) visualization of an anatomical structure (e.g., subcortical anatomy). For example, it is possible to receive a magnetic resonance image, and apply a power regularization convolutional neural network to the MRI. In this manner, it is possible to generate the visualization of the anatomical structure. It is also possible to receive a fast gray matter acquisition T1 inversion recovery (FGATIR) MRI and apply a power regularization convolutional neural network to the FGATIR MRI in order to generate the visualization of the anatomical structure. Further, it is also possible to receive a FGATIR MRI and apply a convolutional neural network to the FGATIR MRI so as to generate the visualization of the anatomical structure.

### CROSS-REFERENCE TO RELATED APPLICATION(S)

This application relates to and claims priority from U.S. Patent Application No. 63/340,391, filed on May 10, 2022, the entire disclosure of which is incorporated herein by reference.

### BACKGROUND INFORMATION AND SUMMARY OF EXEMPLARY EMBODIMENTS

Conventional MRI does not directly visualize specific thalamus, basal ganglia and brainstem structures targeted by functional neurosurgery. Thus, it can be beneficial to provide exemplary systems, methods and computer-accessible medium that can overcome at least some of these limitations.

To that end, it is possible to provide exemplary systems, methods and computer-accessible medium according to exemplary embodiments of the present disclosure, which can use convolutional neural network (CNN) denoising of Fast Gray Matter Acquisition T1 Inversion Recovery (FGATIR) MRI with power spectrum regularization in order to directly visualize subcortical anatomy

Exemplary embodiments of the present disclosure can include exemplary systems, methods and computer-accessible medium which can be configured to employ three dimensional (3D) FGATIR which can utilize a short inversion time to suppress white matter signal and provide unparalleled direct visualization of brainstem and deep gray matter structures. FGATIR though has low signal-to-noise ratio such that a clinically useful high-resolution dataset requires 42-56-minute acquisitions. Such exemplary systems, methods and computer-accessible medium can be used to evaluate several denoising methods and convolutional neural network architectures to optimize the clinical feasibility of FGATIR. The exemplary systems, methods and computer-accessible medium acquired a large training dataset by scanning, e.g., 12 individuals eight times each to generate high SNR averages, to perform supervised learning and unbiased evaluation of denoising performance on real data. The selected exemplary CNN architecture can use a feed-forward residual learning to learn the optimal noise field. Embodiments may compute the mean-squared error loss between an estimated residual from a noisy input image and the predicted residual, regularized by a penalty on the residual power spectrum to minimize over-smoothing. Embodiments may evaluate the results of training both on simulated additive Gaussian noise, Rician noise, and on true noise from the MR system. The exemplary systems, methods and computer-accessible medium can also evaluate the efficacy of denoising the complex valued raw MRI data. The exemplary systems, methods and computer-accessible medium have been used to observe an increase in pSNR from 30 to 42 (30.6%) using a single-average FGATIR acquisition (14 min scan time). This was similar or equivalent to acquiring four averages using a conventional dataset (56 min scan time). Image contrast and quality were evaluated by board certified neuroradiologist and neurosurgeon. Images were considered sufficient for visualization of the structures relevant to functional neurosurgery applications, including MR-guided focused ultrasound of the VIM.

In some exemplary aspects of the exemplary embodiments of the present disclosure, the exemplary procedures described herein relate to method, system and/or a non-transitory computer-accessible medium having stored thereon computer-executable instructions for creating a direct visualization of subcortical anatomy using which a raw magnetic resonance image (MRI) can be received, and a power regularization convolutional neural network can be applied to the raw MRI. For example, the MRI can be Fast Gray Matter Acquisition T1 Inversion Recovery (FGATIR). The FGATIR can be acquired in an accelerated time window decreasing total scan time a reduction by, for example, a factor of \sqrt{2} to 2, depending on the number of averages originally acquired, by eliminating the need for multiple acquired averages. The power regularization CNN can be tuned to provide an amount of regularization. The amount of regularization can be directly related to an amount of denoising performed on the MRI.

In addition or alternatively, there can be an inverse relationship between the amount of regularization and the amount of denoising. The amount of regularization can be selected to (a) prevent the power regularization CNN from minimizing a mean squared error for the MRI, and/or (b) not prevent any denoising by the power regularization CNN. The amount of regularization can be within a range of 0-5. The regularization amount of 5 can be the image with Poisson distributed noise and a regularization amount of 0 is a minimized mean-squared error loss with no regularization applied.

According to exemplary embodiments of the present disclosure, method, system and/or a non-transitory computer-accessible medium having stored thereon computer-executable instructions can be provided, whereas the power regularization CNN can further include a feed-forward residual learning architecture configured to, e.g., determine a mean-squared error loss between an estimated residual from a noisy input image and the predicted residual, and apply a penalty on the residual power spectrum to minimize over-smoothing. The power regularization CNN can target a normalized power spectrum energy level of 1 for all frequencies ranging from 0 Hz to 150 kHz. The output of applying the power regularization CNN to the MRI can be (i) a sharp and denoised MR, and/or a denoised MRI with a residual that has unit energy at all frequencies between 0 Hz to 150 kHz. In additional exemplary embodiments of the present disclosure, methods, systems and/or a non-transitory computer-accessible medium having stored thereon computer-executable instructions can be provided for creating a direct visualization of subcortical anatomy in which, e.g., a fast gray matter acquisition T1 inversion recovery (FGATIR) magnetic resonance image (MRI) can be received, and a power regularization convolutional neural network can be applied to the FGATIR MRI.

According to certain variants of the exemplary embodiments of the present disclosure, the power regularization convolutional neural network can be trained on (i) an FGATIR training data set including a plurality of FGATIR MRI images, (ii) a single known noise level, and/or (iii) a plurality of noise levels.

For example, the plurality of FGATIR MRI images of the FGATIR training data set can be augmented by, e.g., randomly transposing each FGATIR MRI, and/or supplementing with additive white Gaussian noise and/or Rician distributed noise. Alternatively or in addition, each of the plurality of FGATIR MRI images of the FGATIR can be created from, e.g., eight independent averages reconstructed to image space spatially co-registering using a 6 degrees-of-freedom rigid-body transform and averaged together.

According to further exemplary embodiments of the present disclosure, methods, systems and/or a non-transitory computer-accessible medium having stored thereon computer-executable instructions can be provided for a direct visualization of subcortical anatomy by, e.g., receiving a fast gray matter acquisition T1 inversion recovery (FGATIR) magnetic resonance image (MRI), and applying a convolutional neural network to the FGATIR MRI. The convolutional neural network can be a power spectrum convolutional neural network. The convolutional neural network can be trained on (i) an FGATIR training data set including a plurality of FGATIR MRI images, (ii) a single known noise level. and/or (iii) a plurality of noise levels.

According to yet further exemplary embodiments of the present disclosure, methods, systems and/or a non-transitory computer-accessible medium having stored thereon computer-executable instructions can be provided, in which the plurality of FGATIR MRI images of the FGATIR training data set can be augmented by, e.g., randomly transposing each FGATIR MRI, and supplementing with additive white Gaussian noise and/or Rician distributed noise.

For example, each of the plurality of FGATIR MRI images of the FGATIR is created from eight independent averages reconstructed to image space spatially co-registering using a 6 degrees-of-freedom rigid-body transform and averaged together.

These and other objects, features and advantages of the exemplary embodiments of the present disclosure will become apparent upon reading the following detailed description of the exemplary embodiments of the present disclosure, when taken in conjunction with the accompanying claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a flow diagram of an exemplary augmentation pipeline for FGATIR MRI data according to certain exemplary embodiments of the present disclosure;

FIG. 2 is an exemplary model architecture according to certain exemplary embodiments of the present disclosure;

FIG. 3a is an exemplary graph of power spectra of residuals at varying λ;

FIG. 3b is a set of graphs of PSNR, SSIM, MSE, and S3 sharpness as a function of λ;

FIG. 3c is a set of illustrations providing a qualitative comparison of axial FGATIR images at varying regularization levels;

FIG. 4A is a randomized panel of exemplary illustrations evaluated by raters in a blinded fashion;

FIG. 4B is an illustration providing exemplary rankings given by raters to each evaluated level of regularization along with the results of a Kruskal-Wallis test followed by post-hoc Dunn's test to evaluate pairwise statistical effects;

FIG. 5 is a set of exemplary graphs providing an average power spectra of residuals for test data for each trained network and the model-based approach according to exemplary embodiments of the present disclosure;

FIG. 6B is a set of graphs providing exemplary mean ratings and standard deviations for each rater over 168 total images;

FIG. 6A illustrates a set of illustrations of exemplary heatmaps of p-values from nonparametric Kruskal-Wallis followed by post-hoc Dunn's test to evaluate statistical differences between ratings for each image type according to an exemplary embodiment of the present disclosure; and

FIG. 7 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The following description of exemplary embodiments provides non-limiting representative examples referencing numerals to particularly describe features and teachings of different aspects of the present disclosure. The exemplary embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the exemplary embodiments of the present disclosure to such an extent that other implementations, not specifically covered but within the knowledge of a person of skill in the art having read the description of exemplary embodiments, would be understood to be consistent with an application of the exemplary embodiments of the present disclosure.

### Exemplary Methods

Methods, systems and/or a non-transitory computer-accessible medium according to various exemplary embodiments of the present disclosure can be provided to create and/or utilize a convolutional neural network (CNN) that would improve expert-perceived image quality from clinically-feasible FGATIR image acquisitions. Such exemplary methods, systems and/or a non-transitory computer-accessible medium can obtain index standard, high signal-to-noise FGATIR data from volunteers (8 signal averages requiring ˜2 hrs. of scanning using 3T MRI). Such exemplary data can be used to train a 2-channel CNN to denoise single-average FGATIR images (e.g., 12 min acquisition time) using novel power spectrum (PS) regularization to reduce over-smoothing. Using such exemplary methods, systems and/or a non-transitory computer-accessible medium, it is possible to evaluate optimal power spectrum regularization both quantitatively and via rater assessment and then compare the best performing PS-regularized CNN to alternative state-of-the-art denoising methods using both quantitative analysis and rater assessment. This exemplary comparison can be based on models derived from training with a single known noise level (i.e. our original source MRI data) or simulated randomly distributed input image noise levels.

### Exemplary In-Vivo MRI Data Acquisition

Using exemplary methods, systems and/or a non-transitory computer-accessible medium according to exemplary embodiments of the present disclosure, a HIPPA-compliant, IRB-approved can be utilized, that can acquire high-quality, index standard FGATIR data by acquiring 8 averages from 12 individuals without neurological disease (mean age 31.4+/−4.3, 8 male). FGATIR sequence parameters can include TR/TE/TI=3000/2.11/410 ms, nonselective 180-degree inversion pulse, FA=6\hat{ }∘, 288×288 matrix, 230-mm square field-of-view, 192 0.8-mm sagittal slices, bandwidth=455 Hz/pixel, time=12 min 9 sec per average. Subjects can be scanned in 2 sessions (4 individual averages each session) separated by a 15 minute break on the same day.

In one example, for each subject, the 8 averages can be obtained independently, reconstructed to image space, spatially co-registering using a 6 degrees-of-freedom rigid-body transform with FSL-FLIRT (https://fsl.fmrib.ox.ac.uk/), then averaged together. While there is a signal-to-noise penalty for not obtaining multiple averages in k-space prior to Fourier image transform, this approach (e.g., 8 separate 12 min 9 sec scans versus a continuous scan time of 97 min 12 sec) may avoid image degradation from subtle head motion even in cooperative, tolerant and experienced volunteers, and provide individual averages from the same individual for the CNN training (see below). Clean images can be transformed back into each original coordinate space resulting in 96 total high-resolution 3D noisy/clean training pairs (12 subjects, 8 single average images each paired with their respective 8-average dataset). Complex-valued raw data also may also be retained to evaluate the performance of denoising using an even lower noise regime and the effect of denoising magnitude and phase images separately.

### Exemplary Training Data Preprocessing

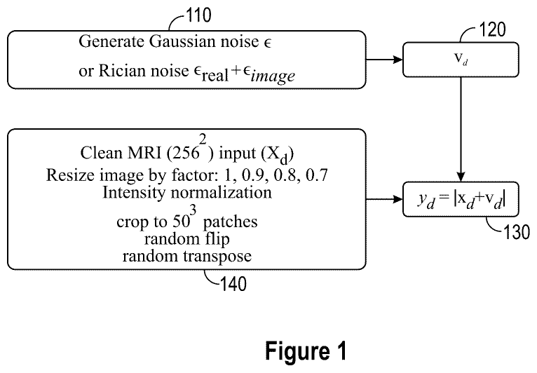

Data augmentation can be used to better generalize the learned network. FIG. 1 shows a flow diagram of an exemplary data augmentation pipeline for FGATIR MRI data according to an exemplary embodiment of the present disclosure. MRI data can be resized, normalized, cropped to 50×50×50 patches, and randomly flipped and/or transposed prior to supplementing with additive white Gaussian Noise (AWGN). The network can be trained using both Gaussian and Rician noise models. Rician distributed noise can be added to simulate noise from the MR system, which is inherently biased because of the magnitude operation during image reconstruction. Rician noise can be simulated by adding 2-channels of Gaussian noise to the clean input data and taking the magnitude over both channels. Noise can be added last to the data augmentation pre-processing pipeline since normalization, rescaling and other basic operations may change the spatial statistics of the Gaussian or Rician noise. Noise can also can be added last so that when computing the loss function the estimated residual image can closely resemble the noisy generated data. The 96 original datasets can be split into 80/10/10 training/test/validation components, where 76 cases may undergo data augmentation and training, 10 cases can be used for network testing, and the final 10 can be used to evaluate network performance quantitatively and compare against other denoising methods.

As illustrated in FIG. 1, there is an augmentation pipeline for the training data preprocessing. This pipeline can begin at 140 with an image, such as a 256×256×160 image, which can then be resized, for instance, by a factor of 1, 0.9, 0.8, 0.7. The resized image can be transformed randomly, patched, and normalized. Either real or complex noise may be generated at step 110 and 120. Then, at step 130 the network input x_{d} can be corrupted by additive noise v_{d} and the absolute value is taken.

### Exemplary Trained Models

Given training set D=[yᵢ, xᵢ]_{i=1 . . . n} where yᵢ, xᵢ denote the ith training pair of noisy and clean images, n represents the number of training images, the goal can be to train a parametric approximation to the posterior of the latent noise field in the input data. For noisy image y, its training pair x can be a simulated clean image obtained by registering and averaging 8 consecutively acquired FGATIR datasets, thus it is not necessarily the exact latent image representation. For this reason, embodiments may also include “clean” data with simulated noise in the training set. Exemplary embodiments may evaluate how noise-level specific and blind noise models perform at reducing Gaussian or Rician distributed noise in FGATIR MRI data.

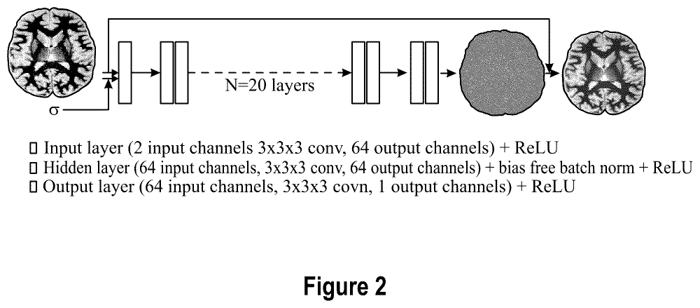

Based on results observed in FFDNet (see, e.g., Zhang, Zuo et al. 2018) embodiments may take a tunable noise level map M as a second input channel to make the denoising model flexible to varying noise levels. Embodiments may also use bias-free batch normalization layers rather than traditional batch-norms—Mohan and colleagues demonstrated that removing bias terms in batch-norm layers can improve a network's generalizability to noise without affecting outcome image quality (Mohan, Kadkhodaie et al. 2019). FIG. 2 shows the fundamental architecture that can be used to train the network.

FIG. 2 illustrates an exemplary model architecture according to an exemplary embodiment of the present disclosure. Blue Boxes denote feature maps 64 channels. This model was implemented with two input channels where the known noise level is included as an input channel to increase blind denoising performance. For a 20-layer network with 2 input channels, the model has ˜667k parameters.

### Exemplary Trained Model Architecture

The exemplary architecture according to the exemplary embodiments of the present disclosure can include, e.g., a discriminative feed-forward convolutional network with two input channels: 1) a patch of noisy data, and 2) the pixel-wise noise-map. The noise map can be or include an image equal in size to the input dataset, where all elements can be set equal to the known added noise level at a given voxel. The first layer can perform convolution+a rectified linear unit (ReLU) to generate 64 (3×3×3) feature maps, followed by 20 layers of convolution (conv)+bias-free batch normalization (Mohan, Kadkhodaie et al. 2019)+ReLU, all with filters of size 3×3×3×64. The additional layer can include another 3×3×3 conv+ReLU with 64 input channel and 1 output channel. Zero-padding can be employed to keep the size of feature maps unchanged after each convolution. Embodiments may adopt a residual network formulation that eases training and delivers better performance (Zhang, Zuo et al. 2018). Using the exemplary embodiments of the present disclosure, denoising can be performed equally well without the use of a residual formulation by increasing the model complexity, however residual learning can be better suited for power spectrum-based regularization, since power spectra are computed directly on residuals (see below). During an exemplary evaluation, the systems, methods and computer-accessible medium according to the exemplary embodiments of the present disclosure can estimate a noise map using the standard deviation of background MRI signal.

### Exemplary Trained Model Optimization

The choice of the regularizer may have an important effect on the quality of the restored image. Equally important can be the ability to efficiently compute the minimum of the overall objective function. Classic residual image denoising under AWG noise, amounts to a loss of the form:

$$L ( θ ) = \frac{1}{2 N} {\sum }_{i = 1}^{N} {‖ R ( {y}_{i} , {σ}_{i} , θ ) - {ε}_{i} ‖}^{2} \qquad ( 1 )$$

Here N refers to the total number of training samples, and i indexes training samples. Training using Eq. 1 can minimize the mean squared error (MSE) between a predicted residual R (defined as the difference between denoised and non-denoised images), and the true noise map ∈ᵢ, and θ denotes the training parameters of the network. Since this network is fully convolutional, it inherits the local connectivity property that the output pixel can be determined by the local noisy input and local noise level. Hence, the trained network naturally may handle spatially-variant noise by specifying a non-uniform noise level map, which is of particular importance to MRI data where signal to noise ratio (SNR) varies spatially over an image, based on both tissue MRI properties sample measured by the specific MRI sequence and coil sensitivity.

To reduce the network from over-smoothing, the systems, methods and computer-accessible medium according to the exemplary embodiments of the present disclosure can utilize a regularization penalty on the loss function. For this over-smoothing penalty, the systems, methods and computer-accessible medium according to the exemplary embodiments of the present disclosure can utilize the power spectrum to encourage the output noise map to be minimally correlated. For independently distributed noise, the systems, methods and computer-accessible medium according to the exemplary embodiments of the present disclosure can expect that a normalized residual (a residual normalized by the local noise level a) will have a power spectrum of 1 at all frequencies. The power spectrum may take the form:

$$Γ ( {k}_{i} ) = \frac{1}{A} ℱ {\{ \frac{R ( {y}_{i} , {σ}_{i} ; θ )}{{σ}_{i}} \}}^{*} ℱ \{ \frac{R ( {y}_{i} , {σ}_{i} ; θ )}{{σ}_{i}} \} \qquad ( 2 )$$

Here F denotes the Fourier transform over image dimensions, e.g., in 2 dimensions (performed slice by slice over a 3D patch), or in 3 dimensions, and A is the total number of voxels in Fourier transform (e.g., the cross-sectional area of the input 2-dimensional patch, or a 3d volume for a 3-dimensional transform). The complete unconstrained optimization problem can be formulated as:

$$arg {min}_{θ} \frac{1}{2 N} {\sum }_{i = 1}^{N} [ \frac{1}{{σ}_{i}^{2}} {‖ R ( {y}_{i} , {σ}_{i} , θ ) - {ϵ}_{i} ‖}^{2} + λ {‖ Γ ( {k}_{i} ) - 1 ‖}^{2} ] \qquad ( 3 )$$

The MSE (mean squared error) term can be normalized by σ² so that the expression is dimensionless, and the two terms scale similarly with image size. Power spectrum regularization can be difficult to train and prone to exploding gradients. Embodiments may apply a filter with Gaussian weights to power spectrum maps to improve training performance and better allow the optimizer to converge to a minimum for both MSE and PS terms.

The exemplary architecture can be trained under several conditions. To measure how regularization changes results compared to MSE loss alone, the network was trained without regularization at single noise levels of AWGN for σ=0.05, 0.1, and 0.2. The same exemplary model was trained on blind noise in the range σ∈[0,0.2], and the regularized PScnn was trained for blind noise in the same regime. To improve the generalizability or external validity of the models the CNN produced (e.g. different SNR than our original data because of different scanner, coil or image resolution), the systems, methods and computer-accessible medium according to the exemplary embodiments of the present disclosure also created data with random spatial noise by combining the 8-average FGATIR data with different amounts of simulated noise. This simulated noise was generated either with a single real channel, or as the magnitude of a real and imaginary channel.

The exemplary network can be optimized using Adaptive Moment Estimation (ADAM) (Kingma and Ba 2014). The network can be trained for a total of 20 epochs and the learning rate can be set to 10⁻³ and decayed to 10⁻⁴ after 10 epochs. Minibatch size can be set to 64 training examples. All models can be trained using pytorch on a Tesla V100 GPU (Nvidia; Santa Clara, Ca.). Training time for each model may take approximately 10 hours.

### Exemplary Experiments

### Exemplary Regularization Tuning

The degree of regularization (A) can determine how much denoising is performed by the network. It can be important to choose a value of λ that is large enough that the network learns to stop minimizing MSE before it begins to oversmooth, but not so large that the network does not perform any denoising at all. Exemplary embodiments generated CNN models using λ=0, 0.5, 0.75, 1.0, 1.25, 1.5, 2.0, 3.0, and 5.0 with single noise value (σ=0.04), then computed residual power spectra, peak SNR (pSNR), Structural Similarity Index Metric (SSIM), MSE and S3 sharpness for each value. S3 sharpness (see, e.g., Vu and Chandler 2009) is a reference-free imaging metric commonly used to assess how sharp an image is (or its inverse, blurring). In this framework λ=0 is an unregularized network trained on MSE loss alone, which can be equivalent to using a denoising convolutional neural network (DnCNN) architecture trained on FGATIR images. Exemplary embodiments therefore may refer to λ=0 models simply as DnCNN.

Based on the quantitative results of this broad range of regularization, the systems, methods and computer-accessible medium according to the exemplary embodiments of the present disclosure then ascertained raters to evaluate an axial FGATIR denoised image of the midbrain at the level of the red nucleus from an individual subject generated using λ=0, 1.0, 1.5, 2.0, 3.0, and 5.0, along with models trained on blind noise for λ=1 and λ=0. Each panel can include a 2×4 arrangement of the above 8 images in random order and there were 12 total panels. Expert raters were two board-certified neuroradiologists familiar with FGATIR contrast, subcortical anatomy, and more than 5 years clinical practice. The two raters were blinded to the source of images and evaluated the images independently from each other. For each validation subjection, each rater ranked the images from best to worst. Overall scores were computed using, e.g.:

$${\sum }_{subjects} {\sum }_{raters} {C}_{j} \qquad ( 4 )$$

where Cⱼ represents the raters ranking (5 being the best quality, 1 worst quality). The cumulatively highest-rated level of regularization over all subjects and raters was chosen to be compared with other denoisers (see next section). Inter-rater variability was measured using the intra-class correlation coefficient (ICC) measuring the degree of consistency among different paired measurements. Pooled rankings from both raters for each training method were compared using non-parametric Kruskal Wallis tests, followed by post-hoc Dunn's test in order to confirm which methods demonstrated significantly improved rankings compared to the rest.
Exemplary Comparison of Power Spectrum CNN (PScnn) with Other Denoisers

Based on the exemplary results described herein, where raters ranked the best performing degree of regularization, embodiments compared denoising convolutional neural networks DnCNN-S (λ=0), DnCNN-B (λ=0 with noise blind training), PScnn-S (λ=1.5), PScnn-B (λ=1.5 with noise blind training), bm4d (Maggioni, Katkovnik et al. 2012), and Gaussian smoothing (smoothing σ=1) evaluated at three image noise levels. These data came from the validation FGATIR dataset, such that these images were not observed by the neural network during training. To validate that the exemplary the systems, methods and computer-accessible medium according to the exemplary embodiments of the present disclosure can operate as expected, for each subject, image slice, and denoising method, the systems, methods and computer-accessible medium according to the exemplary embodiments of the present disclosure determined the power spectra of residuals and measured their deviation from 1 at all frequencies (since power spectra can be used to measure the degree of smoothing introduced by a denoiser). The systems, methods and computer-accessible medium according to the exemplary embodiments of the present disclosure also measured the pSNR, SSIM, and S3 image sharpness for each denoised validation image.

Axial FGATIR images of the midbrain at the level of the red nucleus denoised by these methods were rated by the same 2 experts in blinded, independent manner. Raters observed a total of 14 images per subject (168 images total) in two batches 6 weeks apart (12 images were repeated to assess intra-rater agreement). For each sample image, raters assessed contrast resolution, signal homogeneity, artificiality and overall quality. Contrast resolution was on a 1-4 scale of how easily raters could distinguish adjacent features (i.e. substantia nigra from cerebral peduncle, central tegmental tract from red nucleus and medial lemniscus from surrounding tissue). Signal homogeneity was a measurement (1-4 scale) of the degree of voxel-to-voxel signal variability raters could detect in regions that should be homogeneous. Artificiality was whether the raters felt an image looked computer-generated, smoother or altered in some deviation from typical MRI. Overall clinical quality was the raters' overall assessment of the performance of each technique. For rating scales, higher scores reflected better quality for each scale. Inter-rater reliability was measured using a one-to-one ICC for absolute agreement. A nonparametric Friedman's test was used to detect statistical differences in ratings over all four factors (rating categories). A post-hoc Conover test along with family-wise error correction was used to assess individual significance for each category.

### Exemplary Results

### Exemplary Regularization Tuning

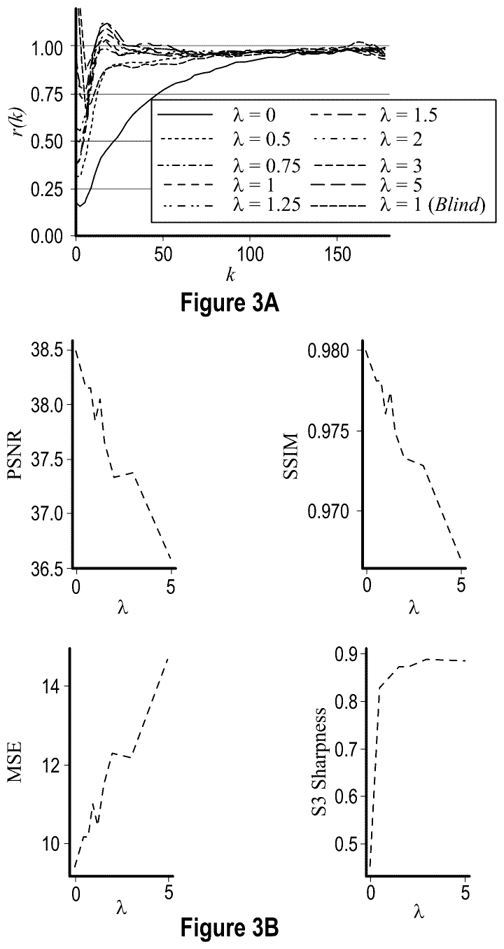

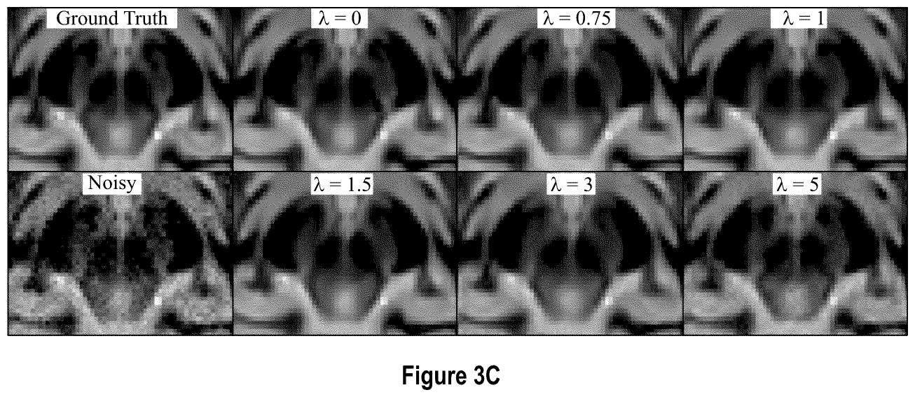

The systems, methods and computer-accessible medium according to the exemplary embodiments of the present disclosure reveal that the performance of the MSE loss regularized by the residual power spectrum may depend heavily on the regularization parameter A. FIG. 3A shows an exemplary graph of residual power spectra for CNNs trained with different penalties at a single noise level (σ=10/255). When λ=0, this is equivalent to the original DnCNN implementation. As A increases, the systems, methods and computer-accessible medium according to the exemplary embodiments of the present disclosure observe an improvement in how the network treats noise at low frequencies, and a corresponding decrease in PSNR and SSIM (see FIG. 3B). This inherent competition between MSE and A can be the expected behavior of this loss function, since the goal of this network is to penalize “over-smoothing” inherent to the network trained on MSE alone. The exemplary graphs of FIG. 3B demonstrates that MSE can choose over-smooth outputs as the global optimum. The systems, methods and computer-accessible medium according to the exemplary embodiments of the present disclosure can show that image sharpness increases with A, and that the ground truth image sharpness corresponds to a high MSE.

The systems, methods and computer-accessible medium according to the exemplary embodiments of the present disclosure can illustrate that the optimal level of regularization occurs when λ˜1.5. FIG. 3C shows exemplary axial FGATIR images through the brainstem at differing regularization levels. As regularization scaling increases, diminished effects of over smoothing and increased effects of noise can be evident. The systems, methods and computer-accessible medium according to the exemplary embodiments of the present disclosure can illustrate that when λ=2 there is over-penalization of the MSE and too much noise remains in the denoised output. For subsequent analyses, The systems, methods and computer-accessible medium according to the exemplary embodiments of the present disclosure can choose a value of A that allows for distinction of fine details without allowing images to remain noisy.

FIGS. 3A-3C, in summary, illustrate analysis of how regularization weighting affects denoising performance for data with simulated Gaussian noise at σ=15/255. A) Power spectra of residuals at varying λ. λ=0 represents no regularization applied. B) PSNR, SSIM, MSE, and S3 sharpness as a function of λ. Minimizing λ maximizes quantitative measures of denoising performance such a PSNR, but also causes over-smoothing. C) Qualitative comparison of axial FGATIR images through the midbrain denoised at varying regularization levels.

### Exemplary Ratings for Regularization

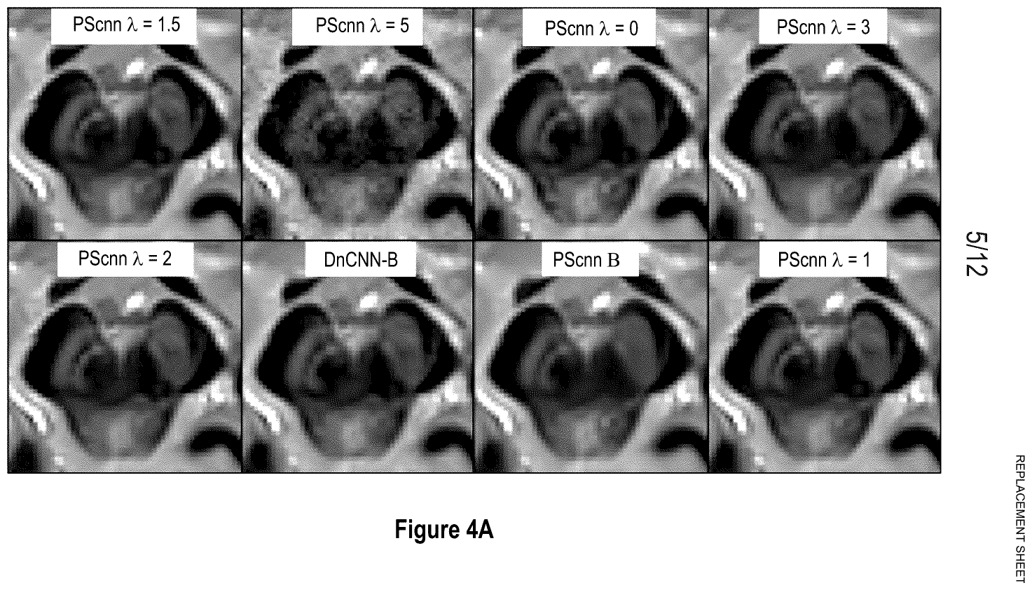

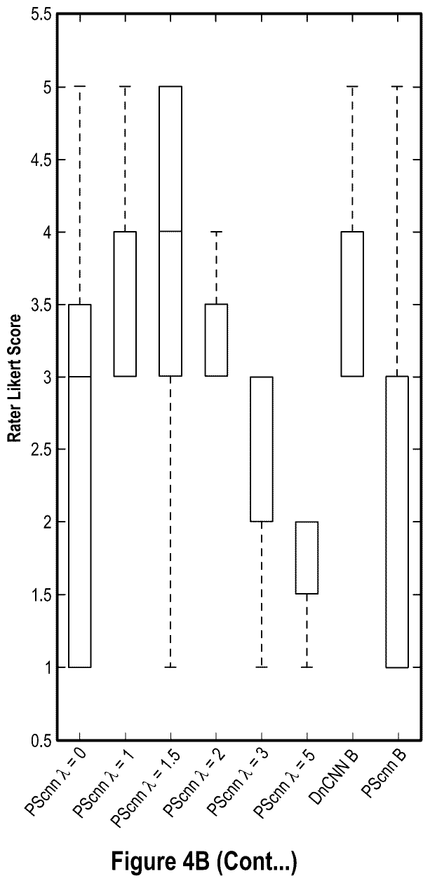

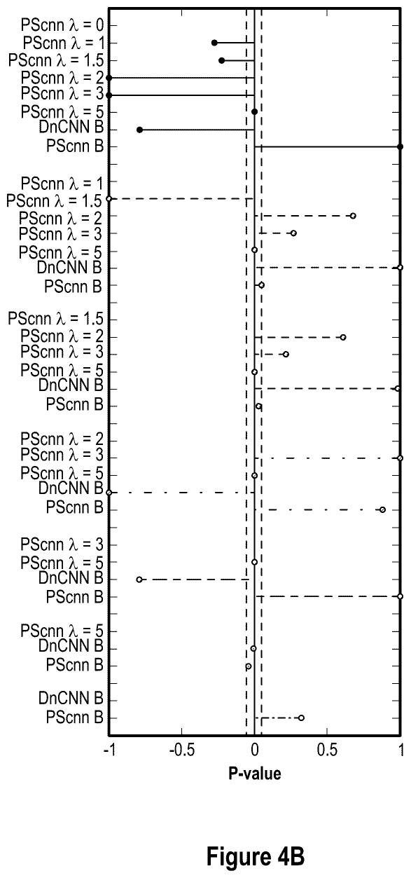

Expert raters observed axial slices through the red nucleus to further determine the optimal value of λ. FIG. 4A shows an example of randomized panels that raters may have observed during the study (of course, raters observed a blinded version of the panel in this figure). Raters also observed cases where blind training was used for both DnCNN (λ=0) and for PScnn (λ=1). FIG. 4B shows an exemplary illustration of the rankings given to different regularizations by raters—after conversion to a 1-5 Likert scale, along with a stem plot showing significant differences between pairs of regularization levels. The highest rated training method was found to be PScnn with λ=1.5, which showed statistically significant improvement only over λ=1.5 and PScnn with blind training data.

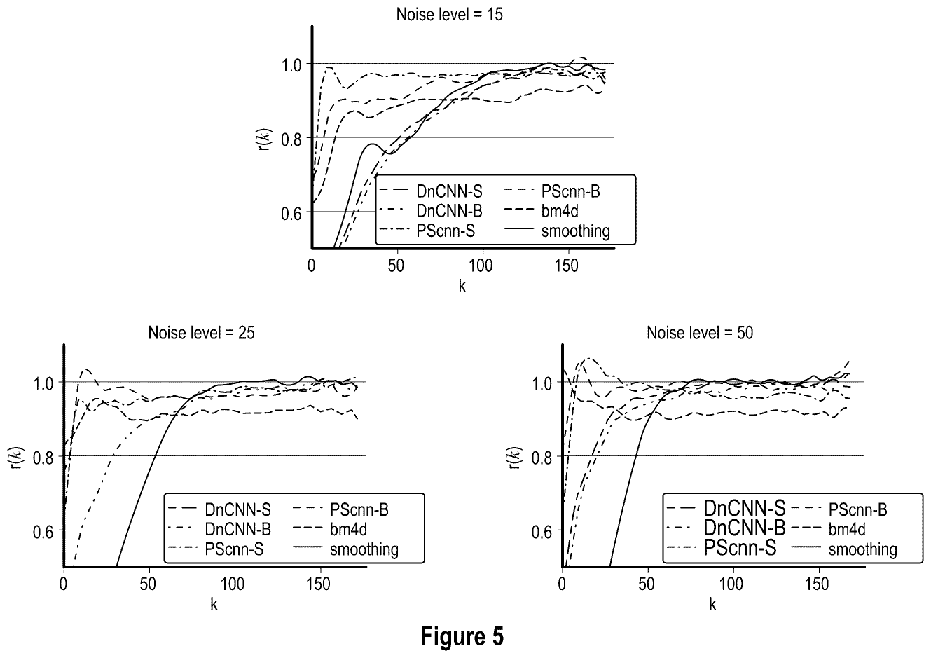

In summary, FIGS. 4A and 5B show A) an example of a randomized panel evaluated by raters in blinded fashion (i.e. not labeled as here), and B) exemplary rankings given by raters to each evaluated level of regularization, along with the results of a Kruskal-Wallis test followed by post-hoc Dunn's test to evaluate pairwise statistical effects. Red dashed lines indicate statistical significance at p=0.05. Raters found that the images with the optimal level of denoising and minimal blurring occurred when λ=1.5. The worst rated images were PScnn with λ=5 which represents severe over-regularization of the loss.

Raters found that the A that provided the best image quality was λ=1.5, followed by λ=1, and λ=0. The images with the worst overall quality came from λ=5. Interestingly, raters gave middling scores to networks that were trained blind. This can be because blind networks tend to generalize to new noise levels through blurring, which is penalized by raters as loss of effective image resolution. The intraclass correlation coefficient over all measurements and all evaluated denoisers between the two raters was 0.54. Statistical comparisons between pooled ratings of different regularization levels identified mainly that a large degree of regularization had a negative impact (λ=1.5 performed significantly worse compared to other methods).

Exemplary Comparison of PScnn with Other Denoisers

To evaluate whether this network works as intended and generates residuals with optimal statistical properties, exemplary embodiments evaluated power spectrum curves for 6 denoisers and three noise levels. The optimal denoiser generates a denoised image with a residual that has unit energy at all frequencies. Low pass filters are known to have low energy at high frequencies due to smoothing over sharp edges. A power-spectrum with energy >1 indicates that the denoiser has added information to the image that was not present in the original (noisy) version. FIG. 5 shows these power spectra for PScnn with λ=0 (equivalent to DnCNN) trained blind and at noise levels 15, 25, and 50, PScnn trained blind and at the same noise levels, along with smoothing and bm4d for reference comparison. PScnn generated residuals with close to optimal energy across all frequencies, while DnCNN generates power spectra that resemble low pass filters. Comparison demonstrates that a power spectrum-based regularization reduced over-smoothing effects.

FIG. 5 illustrates a set of exemplary graphs providing an average power spectra of residuals for test data for each trained network and the model-based approach. PScnn-S refers to a network trained at a specific noise level, PScnn-B refers to the same network train on blind noise, with the single noise level network trained at λ=1.5. Noise specific networks tend to have the most optimal normalized energy densities (closest to 1). For example, bm4d and Gaussian smoothing (with σ=1) was included for comparison with standard model based denoising methods. EXPLAIN performance differences for different noise levels?

Quantitative comparisons of pSNR, SSIM, and S3 sharpness across denoisers and at varying noise levels (see Table 1) demonstrated comparable performance for PScnn. PScnn showed high pSNR and SSIM scores without compromising image sharpness across all noise levels. DnCNN-S had the largest pSNR at noise levels 15 and 50, but had lower sharpness scores at the same noise levels. Bm4d notably had the higher sharpness scores compared to PScnn-S but performed worse in image quality measures. PScnn-B showed considerably higher S3 image sharpness measurements (S3>0.8) across all noise levels, however this was accompanied by the lowest image quality scores.

TABLE 1
Exemplary Quantitative measures of denoising performance for DnCNN-S,
DnCNN-B, PScnn-S, PScnn-B, bm4d, and Gaussian smoothing. pSNR, SSIM,
and S3 sharpness are shown at 3 noise levels (15, 25, and 50) shown
in parentheses. DnCNN-S is equivalent to PScnn trained with λ =
0 at a given noise level. Blind training regime consisted of noise levels
from 5 to 50 for both DnCNN-B and PScnn-B. PScnn methods had the highest
sharpness levels compared to all other evaluated denoising tools.

| | DnCNN-S | DnCNN-B | PScnn-S | PScnn-B | bm4d | smoothing |
|---|---|---|---|---|---|---|
| pSNR (15) | 38.50 | 38.08 | 38.15 | 35.18 | 37.59 | 34.76 |
| pSNR (25) | 32.22 | 33.34 | 33.28 | 30.96 | 32.31 | 30.74 |
| pSNR (50) | 29.90 | 29.66 | 26.93 | 25.48 | 28.41 | 27.45 |
| SSIM (15) | 0.98 | 0.98 | 0.98 | 0.95 | 0.98 | 0.95 |
| SSIM (25) | 0.93 | 0.94 | 0.94 | 0.87 | 0.93 | 0.87 |
| SSIM (50) | 0.87 | 0.86 | 0.72 | 0.64 | 0.83 | 0.74 |
| S3 (15) | 0.45 | 0.43 | 0.46 | 0.81 | 0.50 | 0.22 |
| S3 (25) | 0.60 | 0.46 | 0.62 | 0.86 | 0.69 | 0.05 |
| S3 (50) | 0.44 | 0.68 | 0.92 | 0.96 | 0.86 | 0.02 |

### Exemplary Ratings for Denoiser Comparison

Owing to the inverse relationship between image sharpness and quantitative image quality measurements, embodiments further included an expert rater study to evaluate the qualitative features of each denoising method.

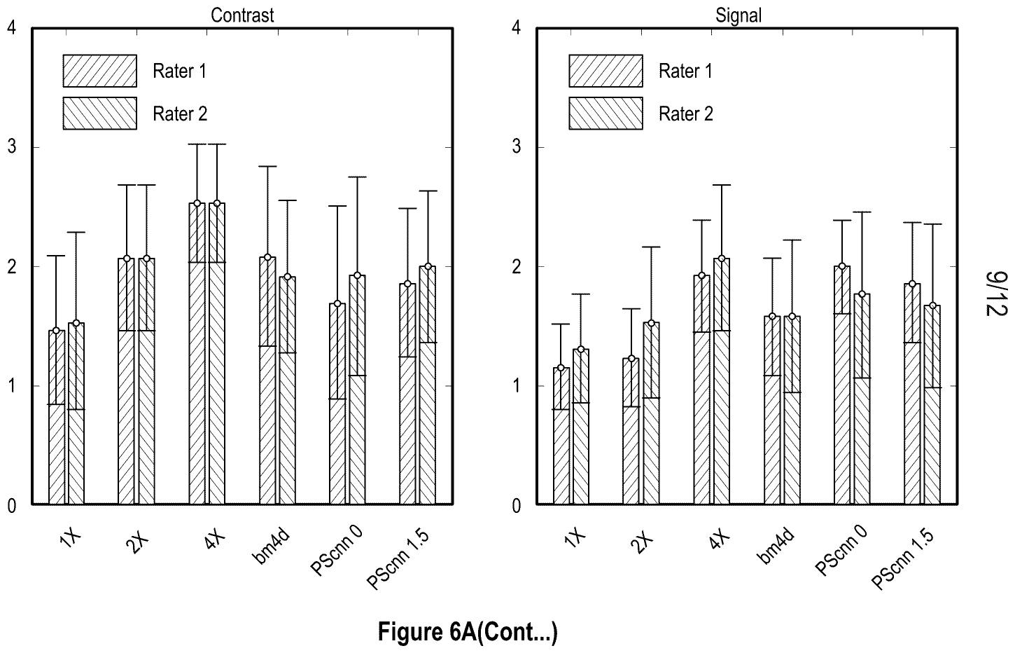

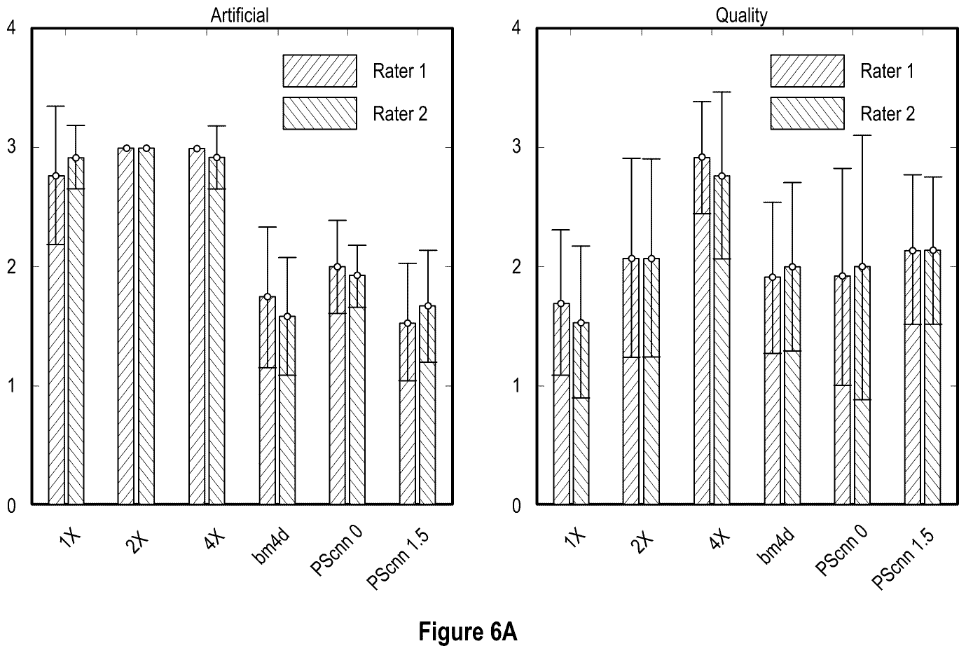

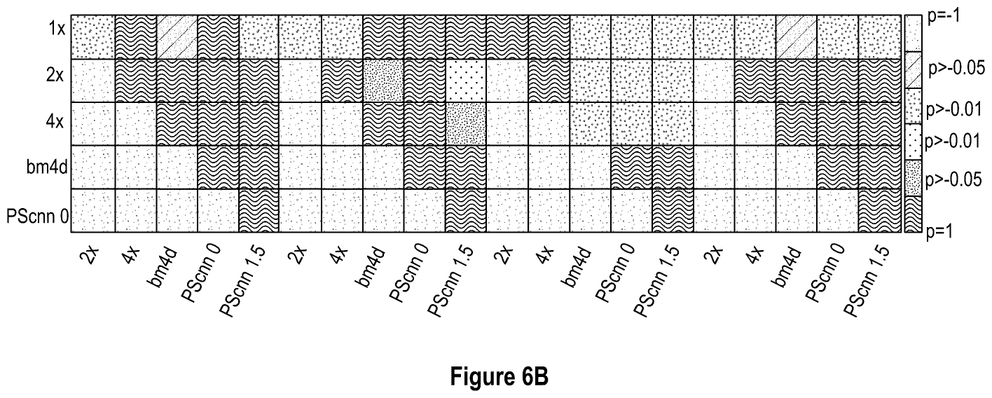

FIG. 6A shows an illustration of a set of mean ratings and standard deviations for each rater over 168 total images. FIG. 6B shows a set of exemplary heatmaps of p-values from nonparametric Kruskal-Wallis followed by post-hoc Dunn's test to evaluate statistical differences between ratings for each image type. Red vs blue indicates the direction of significant difference, dark red/blue shows p<0.01 and light red/blue shows p<0.05.

Both expert raters found that optimal overall image quality came from data that underwent four averages (average ratings of overall quality of 2.93 and 2.71 for each rater). PScnn was rated modestly better than DnCNN for signal contrast and overall quality (both raters gave PScnn 2.76 for overall quality and 2.0 for image contrast). Notably DnCNN was not significantly different from a single average FGATIR image in image contrast owing to a large variance in ratings for this denoiser. All evaluated denoisers were shown to have significantly higher overall image quality compared to a single average FGATIR, with both DnCNN and PScnn having p-values less than 0.01. All denoisers were found to be significantly “artificial” looking according to raters compared to images that were averaged directly to boost SNR.

Raters showed a large degree of consistency and agreement when comparing the effect of denoising to that of averaging. However, rater agreement varied depending on the metric being evaluated. Intraclass correlation coefficients showing the degree of consistency among raters was 85% in contrast resolution, 54% in signal homogeneity, 38% in artificiality, and 71% in overall image quality.

### Exemplary Conclusions and Exemplary Discussion

The systems, methods and computer-accessible medium according to the exemplary embodiments of the present disclosure can provide two fundamental findings. A) Regularized MSE losses have the capacity to decrease blurring introduced during supervised denoising tasks. Exemplary results shown in FIGS. 3A-4B show that the degree of regularization has a direct impact on resulting image sharpness. Expert raters found that images with the optimal tradeoff between quality and sharpness occur with λ˜1.0 . . . 1.5. B) Denoising FGATIR images can facilitate the exemplary visualization of midbrain nuclei that are extremely difficult to see due to the corruption of thermal noise. While other denoisers also allow for the visualization of these structures, PScnn provides images with the best resolution of image features, and the sharpest contrast between adjacent structures (see, e.g., FIGS. 6A and 6B).

Further, exemplary system, method and computer accessible medium according to the exemplary embodiments of the present disclosure can be provided to increase the clinical feasibility of FGATIR data. The systems, methods and computer-accessible medium according to the exemplary embodiments of the present disclosure can accomplish this through supervised CNN denoising, and FIGS. 6A and 6B illustrate the relative improvement to the quality of midbrain images gained through different denoisers, or from averaging k-space data from the scanner. It is evident both qualitatively and quantitatively that the denoising approach developed here can provide SNR improvement to FGATIR images equivalent to acquiring between 2 averages on the scanner, potentially saving up to 32 minutes of scan time per patient.

For an exemplary optimal denoiser, embodiments may anticipate the energy of normalized residuals to be unity at all frequencies, where energy less than 1 can indicate that the filter is removing too much information and greater than 1 indicates the filter can be adding unwanted information. Regularizing the blind network using the power spectrum aids in ensuring unity power spectra. In addition, as SNR increases, the network denoises low spatial frequency information very little, since the data there is already smooth.

As shown in Table 1, PSNR values tend to be unreliable in evaluating the performance of denoising images with real world MRI noise. This is likely an outcome of the fact that there is no true ground truth for data with real world noise. In exemplary embodiments, the “ground truth” for real world noise may come from data that has been registered and averaged together. Therefore, this clean image can have signal features that are derived from other noisy datasets, and likely include some blurring from registration uncertainty. Therefore, pSNR may not be the best measure of performance for real world noise, instead, FIG. 6 shows that for real world noise, the systems, methods and computer-accessible medium according to the exemplary embodiments of the present disclosure can observe optimal performance with either DnCNN or PScnn. Since real world noise varies spatially over FGATIR images, it can indicate that blind denoisers have improved performance, since they are inherently able to adapt to different noise levels depending on the spatial location and observed neuroanatomy in each dataset.

One additional advantage of power spectrum regularization is that it does not explicitly require supervision during training. In exemplary embodiments of the present disclosure, the MSE provides only data fidelity. However, according to other exemplary embodiments of the present disclosure, it is possible to combine PS based loss with Stein's unbiased risk estimator (SURE) (Stein 1981) or MSURE (see, e.g., Ramani, Blu et al. 2008) unsupervised losses to investigate how the power spectrum impacts noise modeling without an a-priori know ground truth MRI dataset.

PScnn denoising gave a low quality single average FGATIR image the quality of ˜2 averages (e.g., factor of 2 increase in SNR) according to expert neuroradiologist evaluation. PScnn performed better in the spectral domain, implying that this denoiser is performing less smoothing, and can be more externally valid for clinical situations. Combining this exemplary CNN with model-based approaches, switching to undersampled data (e.g. compressed sensing), or combining with unsupervised training methods may further increase the effectiveness, efficiency and feasibility of FGATIR for clinical investigations of pathology and functional neurosurgery targeting in subcortical structures.

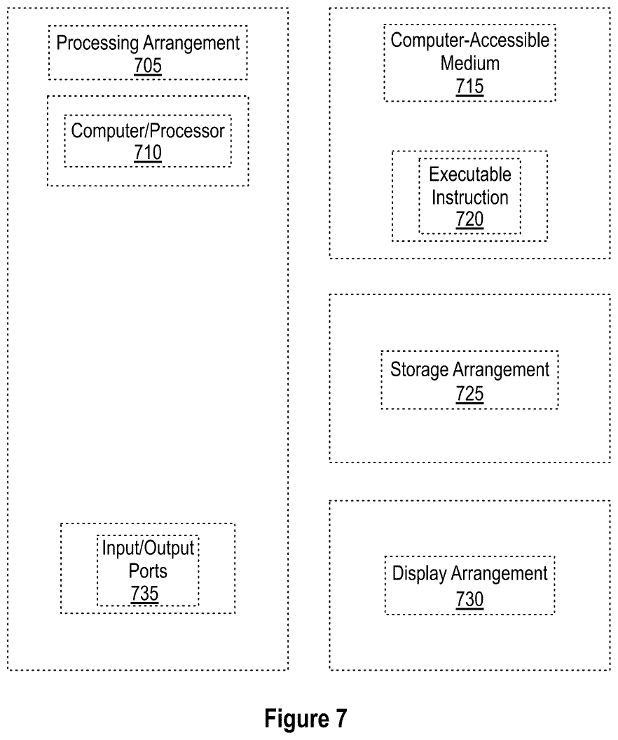

FIG. 7 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 705. Such processing/computing arrangement 705 can be, for example entirely or a part of, or include, but not limited to, a computer/processor 710 that can include, for example one or more microprocessors, and use instructions stored on a computer-accessible medium (e.g., RAM, ROM, hard drive, or other storage device).

As shown in FIG. 7, for example a computer-accessible medium 715 (e.g., as described herein above, a storage device such as a hard disk, floppy disk, memory stick, CD-ROM, RAM, ROM, etc., or a collection thereof) can be provided (e.g., in communication with the processing arrangement 705). The computer-accessible medium 715 can contain executable instructions 720 thereon. In addition or alternatively, a storage arrangement 725 can be provided separately from the computer-accessible medium 715, which can provide the instructions to the processing arrangement 705 so as to configure the processing arrangement to execute certain exemplary procedures, processes, and methods, as described herein above, for example.

Further, the exemplary processing arrangement 705 can be provided with or include an input/output ports 735, which can include, for example a wired network, a wireless network, the internet, an intranet, a data collection probe, a sensor, etc. As shown in FIG. 10, the exemplary processing arrangement 705 can be in communication with an exemplary display arrangement 730, which, according to certain exemplary embodiments of the present disclosure, can be a touch-screen configured for inputting information to the processing arrangement in addition to outputting information from the processing arrangement, for example. Further, the exemplary display arrangement 730 and/or a storage arrangement 725 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference, in their entireties:
1. Buades, A., B. Coll and J.-M. Morel (2005). A non-local algorithm for image denoising. 2005 IEEE computer society conference on computer vision and pattern recognition (CVPR'05), Ieee.
2. Dabov, K., A. Foi, V. Katkovnik and K. Egiazarian (2007). “Image denoising by sparse 3-D transform-domain collaborative filtering.” IEEE Transactions on image processing 16(8): 2080-2095.
3. Ewert, S., P. Plettig, N. Li, M. M. Chakravarty, D. L. Collins, T. M. Herrington, A. A. Kühn and A. Horn (2018). “Toward defining deep brain stimulation targets in MI space: a subcortical atlas based on multimodal MRI, histology and structural connectivity.” Neuroimage 170: 271-282.
4. Kingma, D. P. and J. Ba (2014). “Adam: A method for stochastic optimization.” arXiv preprint arXiv:1412.6980.
5. Li, X., J. Xiao, Y. Zhou, Y. Ye, N. Lv, X. Wang, S. Wang and S. Gao (2020). “Detail retaining convolutional neural network for image denoising.” Journal of Visual Communication and Image Representation 71: 102774.
6. Lozano, A. M., N. Lipsman, H. Bergman, P. Brown, S. Chabardes, J. W. Chang, K. Matthews, C. C. McIntyre, T. E. Schlaepfer and M. Schulder (2019). “Deep brain stimulation: current challenges and future directions.” Nature Reviews Neurology 15(3): 148-160.
7. Maggioni, M., V. Katkovnik, K. Egiazarian and A. Foi (2012). “Nonlocal transform-domain filter for volumetric data denoising and reconstruction.” IEEE transactions on image processing 22(1): 119-133.
8. Mohan, S., Z. Kadkhodaie, E. P. Simoncelli and C. Fernandez-Granda (2019). “Robust and interpretable blind image denoising via bias-free convolutional neural networks.” arXiv preprint arXiv:1906.05478.
9. Niemann, K., R. van den Boom, K. Haeselbarth and F. Afshar (1999). “A brainstem stereotactic atlas in a three-dimensional magnetic resonance imaging navigation system: first experiences with atlas-to-patient registration.” Journal of neurosurgery 90(5): 891-901.
10. Ramani, S., T. Blu and M. Unser (2008). “Monte-Carlo SURE: A black-box optimization of regularization parameters for general denoising algorithms.” IEEE Transactions on image processing 17(9): 1540-1554.
11. Shepherd, T., B. Ades-Aron, M. Bruno, H. Schambra and M. Hoch (2020). “Direct in vivo MRI discrimination of brain stem nuclei and pathways.” American Journal of Neuroradiology 41(5): 777-784.
12. Spiegelmann, R., O. Nissim, D. Daniels, A. Ocherashvilli and Y. Mardor (2006). “Stereotactic targeting of the ventrointermediate nucleus of the thalamus by direct visualization with high-field MRI.” Stereotact Funct Neurosurg 84(1): 19-23.
13. Stein, C. M. (1981). “Estimation of the mean of a multivariate normal distribution.” The annals of Statistics: 1135-1151.
14. Strong, O. S. and A. Elwyn (1948). “Human neuroanatomy.”
15. Sudhyadhom, A., I U. Haq, K. D. Foote, M. S. Okun and F. J. Bova (2009). “A high resolution and high contrast MRI for differentiation of subcortical structures for DBS targeting: the Fast Gray Matter Acquisition T1 Inversion Recovery (FGATIR).” Neuroimage 47 Suppl 2: T44-52.
16. Talairach, J. (1988). “3-dimensional proportional system; an approach to cerebral imaging. co-planar stereotaxic atlas of the human brain.” Thieme: 1-122.
17. Tian, C., Y. Xu, Z. Li, W. Zuo, L. Fei and H. Liu (2020). “Attention-guided CNN for image denoising.” Neural Networks 124: 117-129.
18. Vu, C. T. and D. M. Chandler (2009). S3: a spectral and spatial sharpness measure. 2009 First International Conference on Advances in Multimedia, IEEE.
19. Zhang, K., W. Zuo, Y. Chen, D. Meng and L. Zhang (2017). “Beyond a gaussian denoiser: Residual learning of deep cnn for image denoising.” IEEE transactions on image processing 26(7): 3142-3155.
20. Zhang, K., W. Zuo and L. Zhang (2018). “FFDNet: Toward a fast and flexible solution for CNN-based image denoising.” IEEE Transactions on Image Processing 27(9): 4608-4622.

1. A non-transitory computer-accessible medium having stored thereon computer-executable instructions for creating or providing a visualization of an anatomical structure, wherein, when a computer processor executes the instructions, the computer processor is configured to perform the procedures comprising:
receiving a raw magnetic resonance image (MRI); and
applying a power regularization convolutional neural network (CNN) to the raw MRI so as to generate the visualization of the anatomical structure.

2. The computer-accessible medium of claim 1, wherein the MRI is Fast Gray Matter Acquisition T1 Inversion Recovery (FGATIR).

3. The computer-accessible medium of claim 2, wherein the FGATIR is acquired in an accelerated time window comprising no more than 12 minutes.

4. The computer-accessible medium of claim 1, wherein the power regularization CNN is tuned to provide an amount of regularization.

5. The computer-accessible medium of claim 4, wherein the amount of regularization is directly related to an amount of denoising performed on the MRI.

6. The computer-accessible medium of claim 5, wherein the amount of regularization and the amount of denoising have an inverse relationship therebetween.

7. The computer-accessible medium of claim 4, wherein the amount of regularization is selected to at least one of (a) prevent the power regularization CNN from minimizing a mean squared error for the MRI, or (b) not prevent any denoising by the power regularization CNN.

8. The computer-accessible medium of claim 4, wherein the amount of regularization is between 0 and 5.

9. The computer-accessible medium of claim 8, wherein the regularization amount of 5 is indicative of the MRI with Poisson distributed noise, and wherein a regularization amount of 0 is indicative of a minimized mean-squared error loss without an application of the regularization.

10. The computer-accessible medium of claim 1, wherein the power regularization CNN further comprises a feed-forward residual learning architecture which configures the computer processor to:
determine a mean-squared error loss between an estimated residual from a noisy input image and the predicted residual; and
apply a penalty on a residual power spectrum to minimize over-smoothing.

11. The computer-accessible medium of claim 1, wherein the power regularization CNN targets a power spectrum energy level of 1 for all frequencies in a range between about 0 Hz to about 150 kHz

12. The computer-accessible medium of claim 1, wherein the applying the power regularization CNN to the MRI provides a sharp and denoised MRI.

13. The computer-accessible medium of claim 1, wherein the applying the power regularization CNN to the MRI provides a denoised MRI with a residual that has a unit energy at all frequencies in a range of between about 0 Hz to about 150 kHz.

14. A method for creating or providing a visualization of an anatomical structure, comprising:
receiving a raw magnetic resonance image (MRI); and
applying a power regularization convolutional neural network (CNN) to the raw MRI so as to generate the visualization of the anatomical structure.

15-26. (canceled)

27. A system for creating or providing a visualization of an anatomical structure, comprising:
a computer hardware arrangement configured to:
receive a raw magnetic resonance image (MRI); and
apply a power regularization convolutional neural network (CNN) to the raw MRI so as to generate the visualization of the anatomical structure.

28-39. (canceled)

40. A non-transitory computer-accessible medium having stored thereon computer-executable instructions for creating a visualization of an anatomical structure, wherein, when a computer processor executes the instructions, the computer processor is configured to perform the procedures comprising:
receiving a fast gray matter acquisition T1 inversion recovery (FGATIR) magnetic resonance image (MRI); and
applying a power regularization convolutional neural network to the FGATIR MRI so as to generate the visualization of the anatomical structure.

41. The computer-accessible medium of claim 40, wherein the power regularization convolutional neural network is trained on at least one of (i) an FGATIR training data set comprising a plurality of FGATIR MRI images, (ii) a single known noise level, or (iii) a plurality of noise levels.

42-43. (canceled)

44. The computer-accessible medium of claim 41, wherein the plurality of FGATIR MRI images of the FGATIR training data set are augmented by at least one of:
randomly transposing each FGATIR MRI; or
supplementing each FGATIR MRI with at least one of an additive white Gaussian noise or a Rician distributed noise.

45. (canceled)

46. The computer-accessible medium of claim 41, wherein each FGATIR MRI of the FGATIR is created or provided from eight independent averages reconstructed to image space spatially co-registering using a 6 degrees-of-freedom rigid-body transform and averaged together.

47. A non-transitory computer-accessible medium having stored thereon computer-executable instructions for creating a visualization of an anatomical structure, wherein, when a computer processor executes the instructions, the computer processor is configured to perform the procedures comprising:
receiving a fast gray matter acquisition T1 inversion recovery (FGATIR) magnetic resonance image (MRI); and
applying a convolutional neural network to the FGATIR MRI so as to generate the visualization of the anatomical structure.

48-55. (canceled)
